(19) United States
(12) Patent Application Publication
(10) Pub. No.: US 2013/0045952 A1
(43) Pub. Date: Feb. 21, 2013

(54) ABA Receptor Agonists for Increased Plant Stress Resistance

(71) Applicants: Yong Xu, Guangzhou (CN); Xiaoyin Edward Zhou, Grand Rapids, MI (US); Karsten Melcher, Grand Rapids, MI (US); Huaqiang Eric Xu, Grand Rapids, MI (US)

(72) Inventors: Yong Xu, Guangzhou (CN); Xiaoyin Edward Zhou, Grand Rapids, MI (US); Karsten Melcher, Grand Rapids, MI (US); Huaqiang Eric Xu, Grand Rapids, MI (US)

(73) Assignee: Van Andel Research Institute, Grand Rapids, MI (US)

(21) Appl. No.: 13/588,323

(22) Filed: Aug. 17, 2012

Related U.S. Application Data
(60) Provisional application No. 61/525,337, filed on Aug. 19, 2011.

(51) Int. Cl. A61K 31/4439; A61K 31/197; A61K 31/4402; A61K 31/216; A61P 43/00; A61K 31/341; A61K 31/63; A61K 31/137; A61K 31/4245; A61K 31/403; A61K 31/18

(52) U.S. Cl. 514/155; 514/339; 514/411; 514/562; 514/357; 514/538; 514/602; 514/471; 514/649; 514/364

(57) ABSTRACT

The present invention relates to ABA agonist compounds which increase plant stress resistance. The invention also relates to methods of using these compounds. The invention further relates to compositions and formulations comprising the ABA agonist compounds, and uses thereof.

### CROSS-REFERENCE TO RELATED APPLICATIONS

This application claims priority under 35 U.S.C. §119(e) to and benefit of U.S. Provisional Application Ser. No. 61/525,337, filed Aug. 19, 2011, which is incorporated herein by reference in its entirety.

### TECHNICAL FIELD OF THE INVENTION

The present invention relates to ABA agonist compounds which increase plant stress resistance. The invention also relates to methods of using these compounds. The invention further relates to compositions and formulations comprising the ABA agonist compounds, and uses thereof.

### BACKGROUND OF THE INVENTION

The plant hormone abscisic acid (ABA) controls many important physiological processes, including seed germination, bud dormancy, and adaptive responses to environmental stresses such as drought and salinity. While Glade A of type 2C protein phosphatases (PP2Cs) and subfamily 2 of SNF1-related kinases (SnRK2 kinases) have been established as downstream mediators of ABA signaling, the protein receptors for ABA were not successfully identified for many years because of the high level of receptor redundancy. Through chemical genetics and yeast two-hybrid screening, a new class of START proteins has recently been described as ABA receptors¹⁻³. These receptors are designated as pyrabactin resistance 1 (PYR1) and thirteen members of PYR1-like (PYL) receptors, or as regulatory components of ABA receptors (RCAR). ABA binding to these receptors increases their ability to bind and inhibit PP2Cs, leading to the activation of the SnRK2 kinases, which then activate downstream effectors such as the basic leucine-zipper transcription factors called ABFs/AREBs to switch on stress response programs⁴⁻¹². Recent structural studies have established a conserved gate-latch-lock mechanism underlying ABA perception and signal transduction¹³⁻¹⁷. The apo-ABA receptor contains an open ligand binding pocket. ABA binding induces the closure of the ligand entry gate that allows the receptor to bind to and competitively inhibit PP2Cs. The interactions between PP2Cs and ABA receptors further induce conformational changes that lock the receptor in the closed conformation.

Recent advances in the identification of ABA receptors and improved understanding of the structural mechanisms of ABA receptor binding have created an opportunity for the study of compounds which function as ABA receptor agonists and methods of using such compounds to improve plant stress resistance. Such compounds and methods have the capacity to be highly useful for agriculture, horticulture, and the like by reducing the loss of crops and plants due to environmental stresses.

### SUMMARY OF THE INVENTION

The present invention relates to a method of improving stress resistance in a plant, the method comprising contacting the plant with a compound of Formula I, Formula II or Formula III

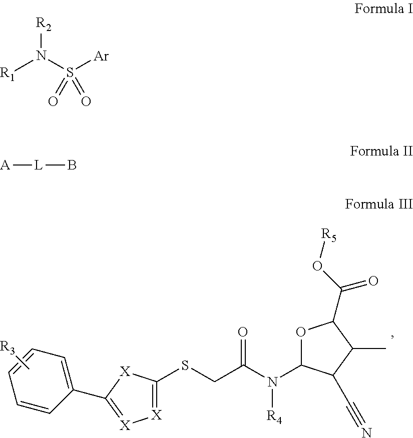

wherein R₁ is a C₁₋₆ branched or straight alkyl group which is optionally and independently substituted with up to 3 of phenoxy, benzyloxy, R′ or R″, each of which is optionally and independently substituted with up to 3 of R′″;

Ar is a phenyl or naphthyl group, which is optionally and independently substituted with up to 3 of R₆;

Each R₆ is independently R′ or R″; or two adjacent R₆ substituents, together with the atoms to which they are attached, form a C₃₋₈ cycloalkyl or heterocycloalkyl ring, which is optionally and independently substituted with up to 3 of R′ or R″;

A and B are each independently phenyl, cyclohexyl or cyclohexenyl, each of which is optionally and independently substituted with up to 4 of R′ or R″;

L is a divalent linear saturated or unsaturated C₄ aliphatic chain which is optionally and independently substituted with R′ or R″, and wherein up to 4 carbon units of the C₄ aliphatic chain are optionally and independently replaced with —C(O)—, —C(S)—, —NR″— or —O—;

R₃ is R′ or R″;

R₂, R₄, and R₅ are each independently R″;

Each X is independently N, NH, N—C₁₋₄ alkyl, O, S, or CH;

Each R′ is independently hydrogen, halo, oxo, —OR″, —C(O)R″, —C(O)OR″, —C(O)NR″₂, —SR″, —S(O)₂R″, —S(O)₂OR″, or —S(O)₂NR″₂;

Each R″ is independently absent, hydrogen, C₁₋₆ alkyl, C₆ heteroaryl or heterocycloalkyl, each of which is optionally and independently substituted with up to 3 of R′″;

Each R′″ is independently hydrogen, halo, oxo, OH, NH₂, NO₂, COOH, C₁₋₄ alkyl, or C₁₋₄ haloalkyl;

with the proviso that the compound is not pyrabactin:

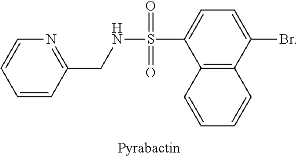 [SMILES: O=S(=O)(NCc1ccccn1)c1ccc(Br)c2ccccc12]

The invention also relates to a method of improving resistance to stress in a plant by contacting the plant with a compound of the invention, or a composition or formulation as described herein.

### BRIEF DESCRIPTION OF THE DRAWINGS

FIG. 1 is a graphical representation of the stimulation of the interaction between 100 nM His6-PYR1 and 100 nM biotin-PP2C in the presence or absence of 100 μM agonist (compounds 1, 2, 3, 4, 11 and 12) determined by AlphaScreen luminescence proximity assay.

FIG. 2 is a graphical representation of the agonist-dependent (compounds 1, 2, 3, and 11) inhibition of phosphatase activity of 200 nM PP2C incubated with 400 nM.

FIG. 3 shows the inhibition of Arabidopsis thaliana seed germination in the absence or presence of 32 or 64 μM agonist (compounds 1, 2, 3, 4, 11 and 12).

FIGS. 4A-4I are graphical representations of the dose-response curves showing the dependence of the interaction between 100 nM His6-PYR1 and 100 nM biotin-PP2C on the presence of 0-100 μM agonists determined by AlphaScreen luminescence proximity assay. A) data for ABI1 binding signal for compound 1; B) data for ABI1 binding signal for compounds 2 and 11; C) data for ABI1 binding signal for compound 3; D) data for ABI2 binding signal for compound 1; E) data for ABI2 binding signal for compounds 2 and 11; F) data for ABI2 binding signal for compound 3; G) data for HAB1 binding signal for compound 1; H) data for HAB1 binding signal for compounds 2 and 11; I) data for HAB1 binding signal for compound 3.

FIG. 5 is a graphical representation of the stimulation of the interaction between 100 nM His6-PYR1 and 100 nM biotin-HAB1 in the presence or absence of 100 μM agonist (compounds 5, 6, 7, 8, 9, 10, 13, 14, and 15) determined by AlphaScreen luminescence proximity assay.

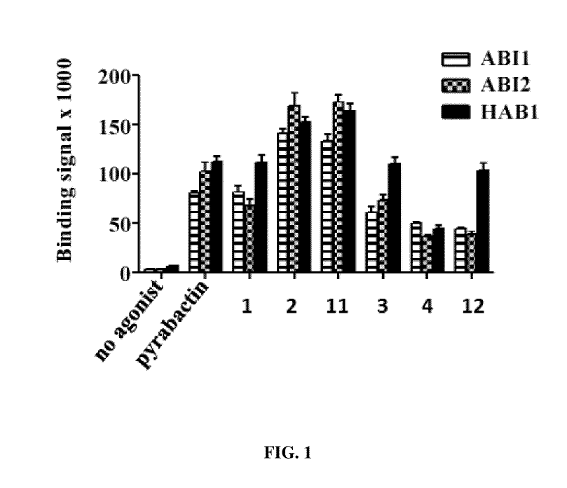

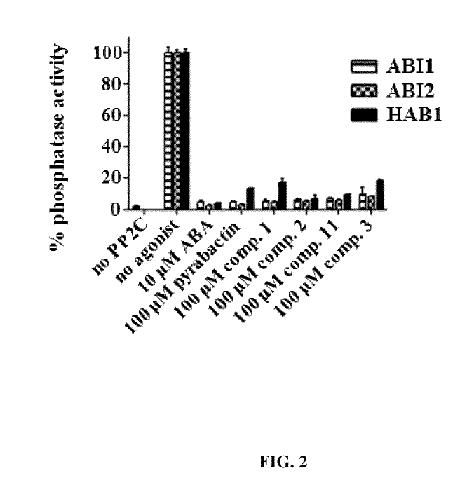

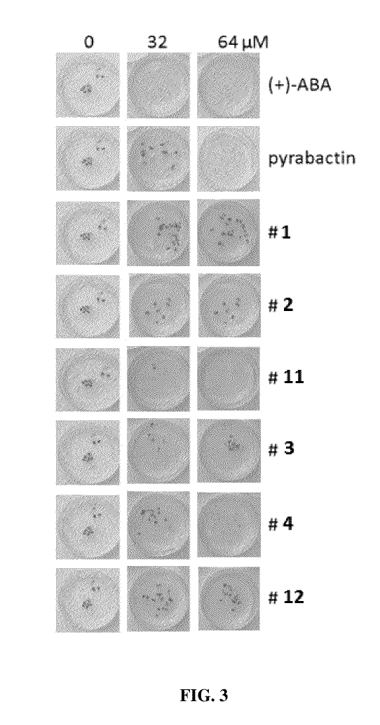

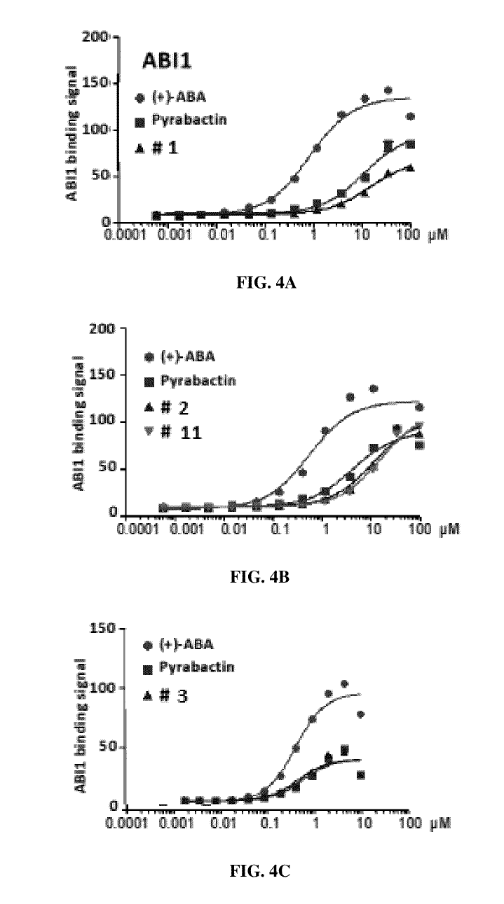

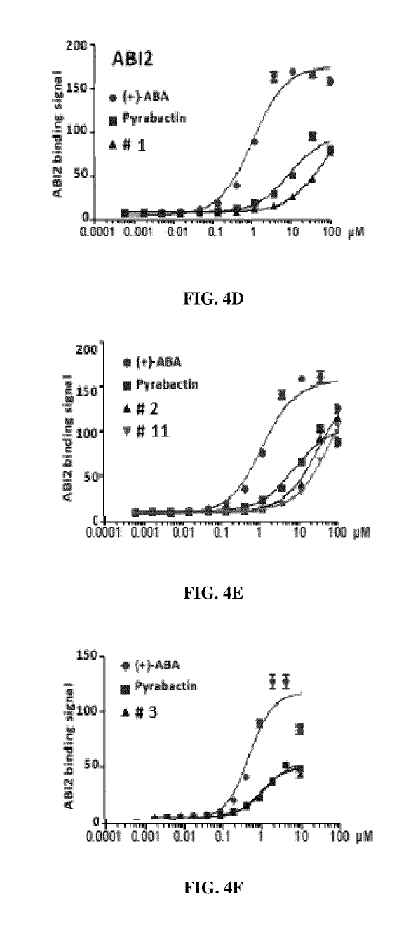

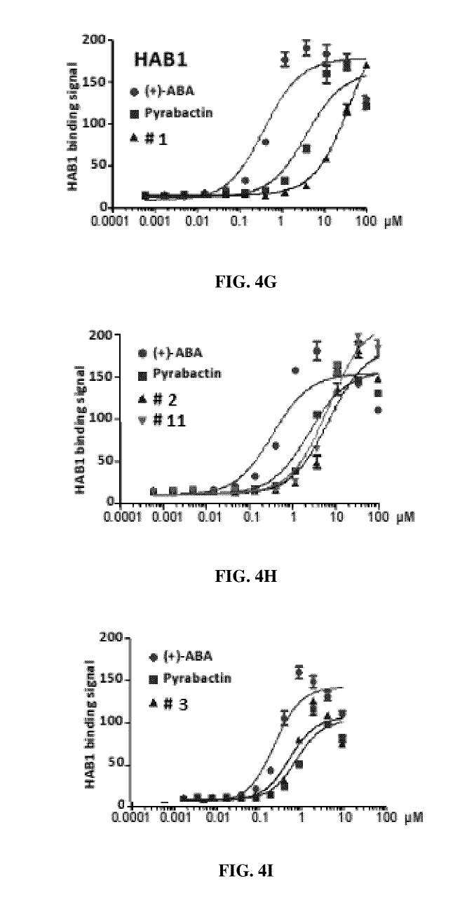

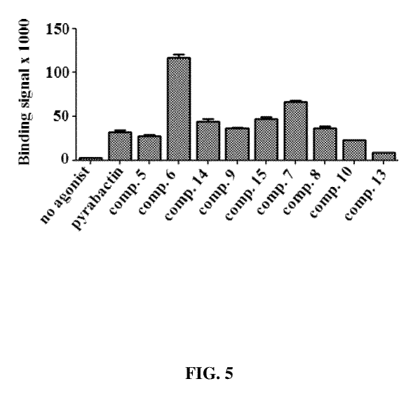

These figures are provided by way of example and are not intended to limit the scope of the invention.

### DETAILED DESCRIPTION

### Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

As described herein, compounds of the invention may optionally be substituted with one or more substituents, such as are illustrated generally above, or as exemplified by particular classes, subclasses, and species of the invention.

As used herein the term “aliphatic’ encompasses the terms alkyl, alkenyl, alkynyl.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-12 (e.g., 2-8, 2-6, or 2-4) carbon atoms and at least one double bond. Like an alkyl group, an alkenyl group can be straight or branched. Examples of an alkenyl group include, but are not limited to allyl, isoprenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-12 (e.g., 2-8, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl”, “aralkoxy”, or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “carbocycle” or “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, cyclopentenyl, bicyclo[2.2.2]octenyl, or bicyclo[3.3.1]nonenyl.

A cycloalkyl or cycloalkenyl group can be optionally substituted with one or more substituents such as phosphor, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)-], sulfanyl [e.g., alkyl-S—], sulfoxy, urea, thiourea, sulfamoyl, sulfamide, oxo, or carbamoyl.

As used herein, the term “heterocycle” or “heterocycloaliphatic” encompasses a heterocycloalkyl group and a heterocycloalkenyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety to form structures, such as tetrahydroisoquinoline, which would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phosphor, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl.

Without limitation, monocyclic heteroaryls include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizinyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl, and (haloalkyl)heteroaryl [e.g., trihaloalkylheteroaryl].

A “heteroaraliphatic” (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicaliphatic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where Rx and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z}, wherein R_{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S—R^{X} when used terminally and —S— when used internally, wherein Rx has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein Rx has been defined above. Exemplary sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Exemplary sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refer to —C(O)—.

As used herein, an “oxo” refers to ═O.

As used herein, a “hydroxyl” refers to —OH.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

In general, the term “vicinal” refers to the placement of substituents on a group that includes two or more carbon atoms, wherein the substituents are attached to adjacent carbon atoms.

In general, the term “geminal” refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A branched aliphatic chain is a straight aliphatic chain that is substituted with one or more aliphatic groups. The term aliphatic chain includes alkyl chains, alkenyl chains, and alkynyl chains, where alkyl, alkenyl, and alkynyl are defined above.

The phrase “optionally substituted” is used interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R₁, R₂, R₃, R₄, R₅, R₆, R′, R″, R′″, X, A and B and other variables contained in formulae described herein encompass specific groups, such as alkyl and aryl. Unless otherwise noted, each of the specific groups for the variables can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

In general, the term “substituted,” whether preceded by the term “optionally” or not, refers to the replacement of hydrogen radicals in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible,” as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention. For Example, Compound 10 below can be present in the keto or enol form:

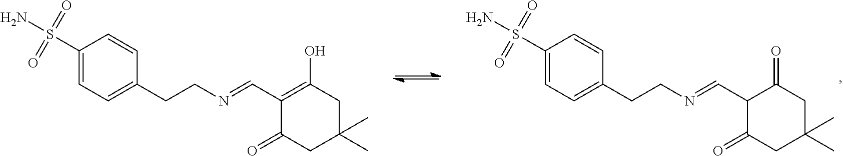 [SMILES: CC1(C)CC(=O)C(/C=N/CCc2ccc(S(N)(=O)=O)cc2)=C(O)C1 ; CC1(C)CC(=O)C(/C=N/CCc2ccc(S(N)(=O)=O)cc2)C(=O)C1]

or in the enolate form as shown in Table 1.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention.

### Compounds

In one aspect, the present invention provides a method of improving stress resistance in a plant, the method comprising contacting the plant with a compound of Formula I, Formula II or Formula III

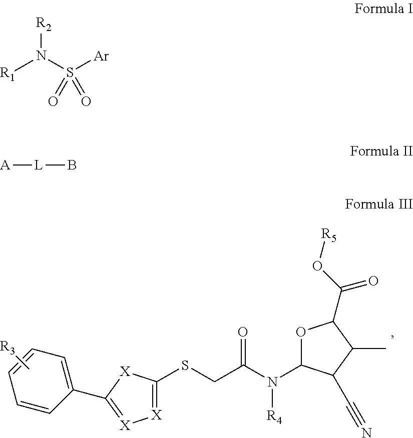

wherein R₁ is a C₁₋₆ branched or straight alkyl group which is optionally and independently substituted with up to 3 of phenoxy, benzyloxy, R′ or R″, each of which is optionally and independently substituted with up to 3 of R′″;

Ar is a phenyl or naphthyl group, which is optionally and independently substituted with up to 3 of R₆;

Each R₆ is independently R′ or R″; or two adjacent R₆ substituents, together with the atoms to which they are attached, form a C₃₋₈ cycloalkyl or heterocycloalkyl ring, which is optionally and independently substituted with up to 3 of R′ or R″;

A and B are each independently phenyl, cyclohexyl or cyclohexenyl, each of which is optionally and independently substituted with up to 4 of R′ or R″;

L is a divalent linear saturated or unsaturated C₄ aliphatic chain which is optionally and independently substituted with R′ or R″, and wherein up to 4 carbon units of the C₄ aliphatic chain are optionally and independently replaced with —C(O)—, —C(S)—, —NR″— or —O—;

R₃ is R′ or R″;

R₂, R₄, and R₅ are each independently R″;

Each X is independently N, NH, N—C₁₋₄ alkyl, O, S, or CH;

Each R′ is independently hydrogen, halo, oxo, —OR″, —C(O)R″, —C(O)OR″, —C(O)NR″₂, —SR″, —S(O)₂R″, —S(O)₂OR″, or —S(O)₂NR″₂;

Each R″ is independently absent, hydrogen, C₁₋₆ alkyl, C₆ heteroaryl or heterocycloalkyl, each of which is optionally and independently substituted with up to 3 of R′″;

Each R′″ is independently hydrogen, halo, oxo, OH, NH₂, NO₂, COOH, C₁₋₄ alkyl, or C₁₋₄ haloalkyl;

with the proviso that the compound is not pyrabactin:

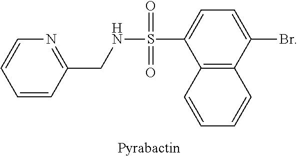 [SMILES: O=S(=O)(NCc1ccccn1)c1ccc(Br)c2ccccc12]

In some embodiments, R₁ is heteroaryl alkyl, alkoxy carbonyl alkyl, heterocycloalkyl alkyl, alkylthio (carboxy)alkyl, aryl alkoxycarbonyl alkyl, alkylamino carbonyl (carboxy)alkyl, dialkylamino carbonyl (carboxy)alkyl or aryloxy alkyl.

In some further embodiments, R₁ is

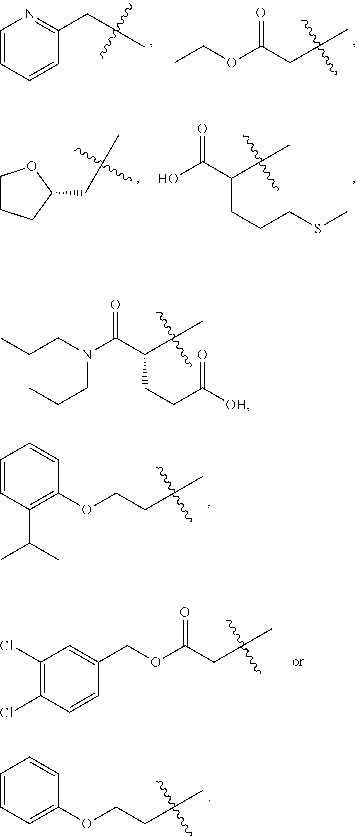

In some embodiments, Ar is phenyl, alkylphenyl, naphthyl, halonaphthyl, alkylnaphthyl, or a substituent having the formula AR′

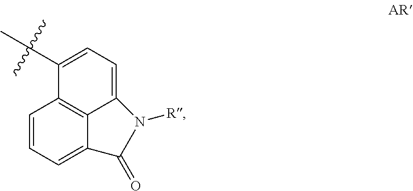

wherein R″ is defined above.

In some embodiments, Ar is a substituent having the formula AR′

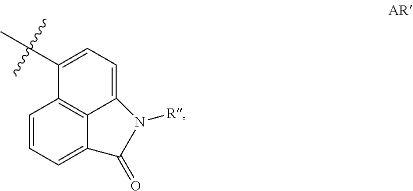

wherein R″ is defined above.

In some further embodiments, Ar is selected from

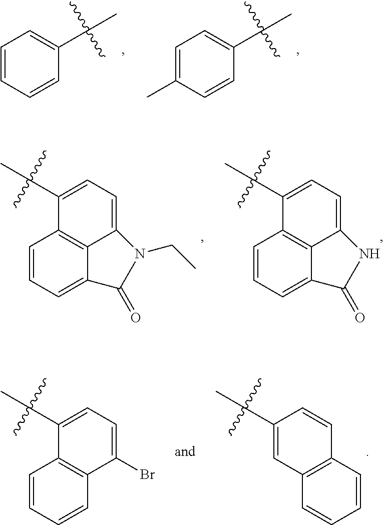

In some embodiments, A and B are each independently alkyl (carboxy)phenyl, halophenyl, aminosulfonyl phenyl, alkoxy phenyl, alkylphenyl, dihalophenyl, acetylphenyl, dioxo dialkyl cyclohexane or hydroxyl oxo dialkyl cyclohexene.

In some further embodiments, A and B are each independently

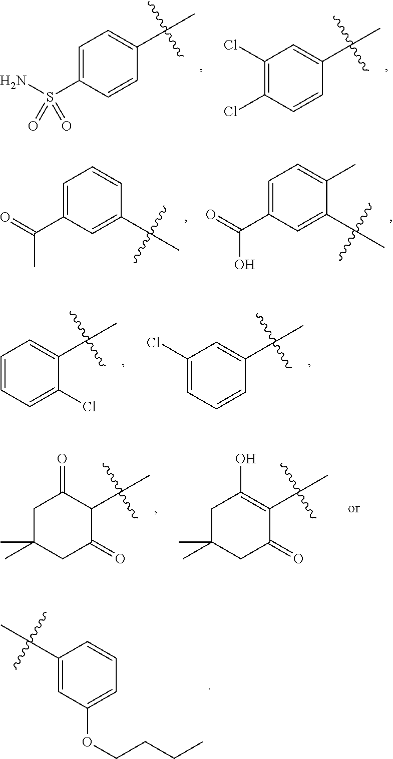

In some embodiments, L is a divalent linear saturated or unsaturated C₄ aliphatic chain which is optionally substituted with R′ or R″, and wherein up to 4 carbon units of the C₄ aliphatic chain are optionally and independently replaced with —C(O)—, —C(S)—, or —NH—. In a further embodiment, two carbon units of L are replaced with —NH—, one carbon unit is replaced with —C(O)—, and one carbon unit is replaced with —C(S)—. In a further embodiment, L is

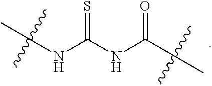 [SMILES: *NC(=S)NC(*)=O]

In some embodiments, L is a divalent linear unsaturated C₄ aliphatic chain which is optionally substituted with R′ or R″, and wherein one carbon unit of the C₄ aliphatic chain is optionally replaced with —NH— or —N═ and one carbon is optionally replaced with C(O). In a further embodiment, L is

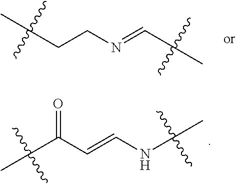 [SMILES: */C=N/CC* ; *N/C=C/C(*)=O]

In some embodiments, R₃ is C₁₋₆ alkoxy; each X is N or O; R₄ is hydrogen and R₅ is C₁₋₆ alkyl. In some further embodiments, R₃ is ethoxy; each X is N or O; R₄ is hydrogen and R₅ is ethyl.

In another aspect, the compound of formula I is also a compound of formula Ia

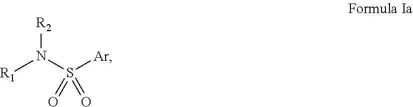

wherein R₁ is alkoxy carbonyl alkyl, heterocycloalkyl alkyl, alkylthio (carboxy)alkyl, aryl alkoxycarbonyl alkyl, alkylamino carbonyl (carboxy)alkyl, dialkylamino carbonyl (carboxy)alkyl or aryloxy alkyl, and R₂ and Ar are defined as above.

In some further embodiments of this aspect, R₁ is

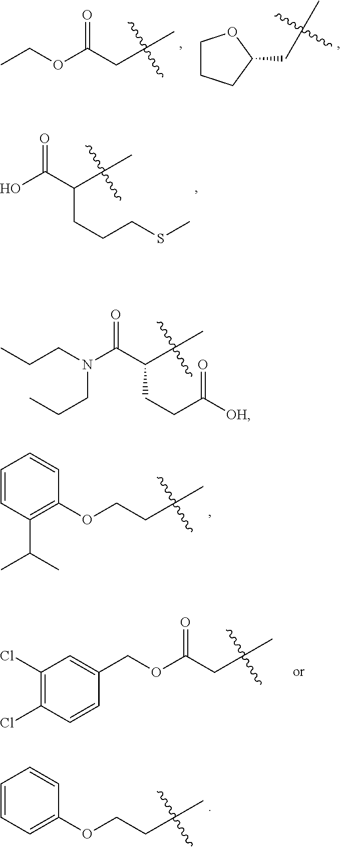

In another aspect, the compound of formula I is also a compound of formula Ib

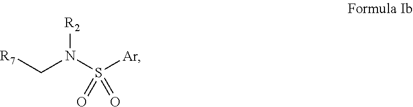

wherein R₇ is 2-pyridyl, 3-pyridyl, 4-pyridyl, 2,6-pyrimidinyl or 3,5-pyrimidinyl; and R₂ and Ar are as defined above.

In another aspect, the compound of formula I is also a compound of formula Ic

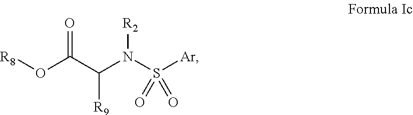

wherein R₈ and R₉ are each independently hydrogen, C₁₋₄ alkyl or benzyl, and wherein the alkyl and benzyl are each optionally and independently substituted up to 3 R′ or R″; and R′, R″, R₂ and Ar are as defined above.

In another aspect, the compound of formula I is also a compound of formula Id

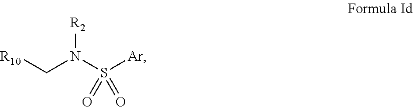

wherein R₁₀ is 2-tetrahydrofuranyl, 3-tetrahydrofuranyl, 2-tetrahydrothiophenyl, 3-tetrahydrothiophenyl, 2 pyrrolidinyl or 3-pyrrolidinyl, each of which is optionally substituted with R′ or R″; and R′, R″, R₂ and Ar are as defined above.

In another aspect, the compound of formula I is also a compound of formula Ie

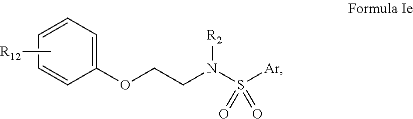

wherein R₁₂ is phenyl optionally substituted with R′ or R″; and R′, R″, R₂ and Ar are as defined above.

In another aspect, the compound of formula II is also a compound of formula IIa

A-L-B Formula IIa

wherein L is a divalent linear unsaturated C₄ aliphatic chain which is optionally substituted with R′ or R″, and wherein one carbon unit of the C₄ aliphatic chain is optionally and independently replaced with —NH— or —N═ and one carbon is optionally replaced with C(O); and R′, R″, A and B are defined as above.

In another aspect, the compound of formula II is also a compound of formula IIb

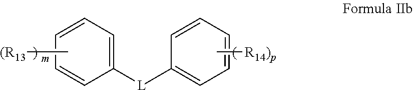

wherein R₁₃ and R₁₄ are each independently hydrogen, halo, —S(O)₂NH₂, C₁₋₄ alkyl, C₁₋₄ alkoxy, or C₁₋₄ acyl; m and p are each independently 0-3; and L is defined as above.

In another aspect, the compound of formula II is also a compound of formula IIc

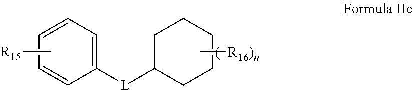

wherein R₁₅ is —S(O)₂NH₂; and each R₁₆ is independently hydrogen, oxo, hydroxyl or C₁₋₄ alkyl; n is 0-3; and L is defined as above.

In a further embodiment, L is

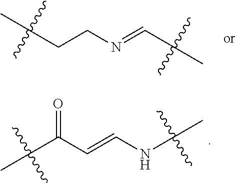 [SMILES: */C=N/CC* ; *N/C=C/C(*)=O]

In some embodiments, the compound of formula I is a compound of formula Ia and the compound of formula II is a compound of formula IIa.

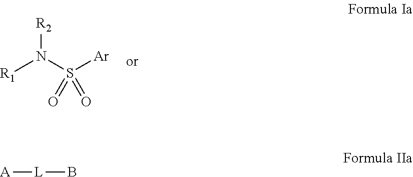

wherein R₁ is alkoxy carbonyl alkyl, heterocycloalkyl alkyl, alkylthio (carboxy)alkyl, aryl alkoxycarbonyl alkyl, alkylamino carbonyl (carboxy)alkyl, dialkylamino carbonyl (carboxy)alkyl or aryloxy alkyl;

Ar is phenyl or naphthyl group, which is optionally and independently substituted with up to 3 of R₆;

Each R₆ is independently R′ or R″; or two adjacent R₆ substituents, together with the atoms to which they are attached, form a C₃₋₈ cycloalkyl or heterocycloalkyl ring, which is optionally and independently substituted with up to 3 of R′ or R″;

A and B are each independently phenyl, cyclohexyl or cyclohexenyl, each of which is independently and optionally substituted with up to 4 R′ or R″ substituents;

L is a divalent linear unsaturated C₄ aliphatic chain which is optionally substituted with R′ or R″, and wherein one carbon unit of the C₄ aliphatic chain is optionally and independently replaced with —NH— or —N═ and one carbon is optionally replaced with C(O);

R₂ is R″;

Each R′ is independently hydrogen, halo, oxo, —OR″, —C(O)R″, —C(O)OR″, —C(O)NR″₂, —SR″, —S(O)₂R″, —S(O)₂OR″, or —S(O)₂NR″₂;

Each R″ is independently absent, hydrogen, C₁₋₆ alkyl, aryl, heteroaryl, cycloalkyl or heterocycloalkyl, each of which is optionally and independently substituted with up to 3 of R′″;

Each R′″ is independently hydrogen, halo, oxo, OH, NH₂, NO₂, COOH, C₁₋₄ alkyl, or C₁₋₄ haloalkyl.

Exemplary compounds of the present invention include, but are not limited to, those illustrated in Tables 1-3, below.

TABLE 1
Exemplary compounds of formula I

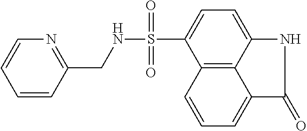 [SMILES: O=C1Nc2ccc(S(=O)(=O)NCc3ccccn3)c3cccc1c23]

1

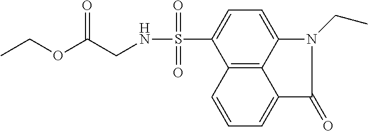 [SMILES: CCOC(=O)CNS(=O)(=O)c1ccc2c3c(cccc13)C(=O)N2CC]

2

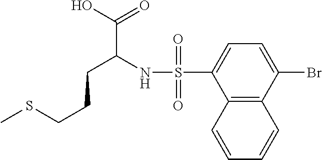

3

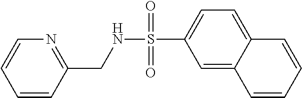 [SMILES: O=S(=O)(NCc1ccccn1)c1ccc2ccccc2c1]

4

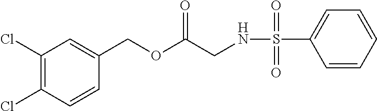 [SMILES: O=C(CNS(=O)(=O)c1ccccc1)OCc1ccc(Cl)c(Cl)c1]

6

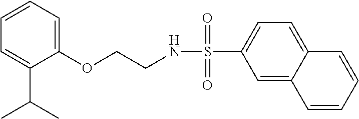 [SMILES: CC(C)c1ccccc1OCCNS(=O)(=O)c1ccc2ccccc2c1]

7

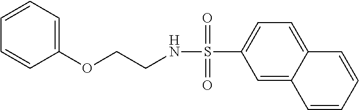 [SMILES: O=S(=O)(NCCOc1ccccc1)c1ccc2ccccc2c1]

8

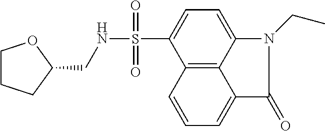 [SMILES: CCN1C(=O)c2cccc3c(S(=O)(=O)NC[C@@H]4CCCO4)ccc1c23]

11

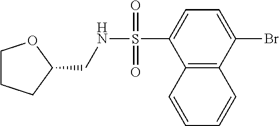 [SMILES: O=S(=O)(NC[C@@H]1CCCO1)c1ccc(Br)c2ccccc12]

12

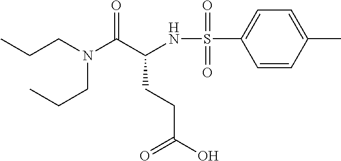 [SMILES: CCCN(CCC)C(=O)[C@@H](CCC(=O)O)NS(=O)(=O)c1ccc(C)cc1]

13

TABLE 2
Exemplary compounds of formula II

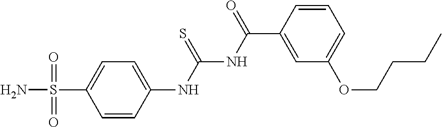 [SMILES: CCCCOc1cccc(C(=O)NC(=S)Nc2ccc(S(N)(=O)=O)cc2)c1]

9

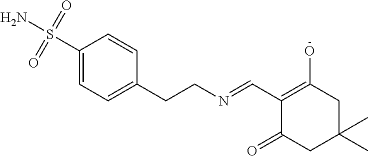 [SMILES: CC1(C)CC(=O)C(/C=N/CCc2ccc(S(N)(=O)=O)cc2)=C(O)C1]

10

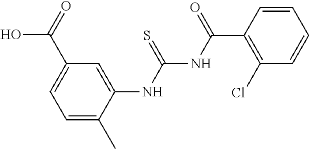 [SMILES: Cc1ccc(C(=O)O)cc1NC(=S)NC(=O)c1ccccc1Cl]

14

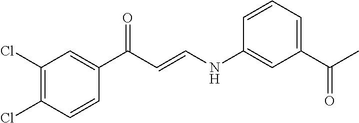 [SMILES: CC(=O)c1cccc(N/C=C/C(=O)c2ccc(Cl)c(Cl)c2)c1]

15

TABLE 3
Exemplary compound of formula III

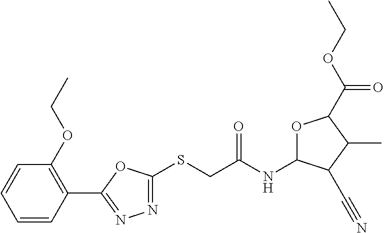 [SMILES: CCOC(=O)C1OC(NC(=O)CSc2nnc(-c3ccccc3OCC)o2)C(C#N)C1C]

5

### Compositions and Formulations

In these compositions, the active ingredient is employed in pure form, a solid active ingredient for example in a specific particle size, or, preferably, together with at least one of the auxiliaries conventionally used in the art of formulation, such as extenders, for example solvents or solid carriers, or such as surface-active compounds (surfactants).

Examples of suitable solvents are: unhydrogenated or partially hydrogenated aromatic hydrocarbons, preferably the fractions C₈₋₁₂ of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorptive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues.

Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature.

Suitable non-ionic surfactants are, especially, polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediamino polypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxy polyethoxyethanol, castor oil polyglycol ether, polypropylene glycol/polyethylene oxide adducts, tributylphenoxy polyethoxyethanol, polyethylene glycol or octylphenoxy polyethoxyethanol. Also suitable are fatty acid esters of polyoxyethylene sorbitan, such as polyoxyethylene sorbitan trioleate.

The cationic surfactants are, especially, quaternary ammonium salts which generally have at least one alkyl radical of approximately 8 to approximately 22 C atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzyl bis(2-chloroethyl/thylammonium bromide. Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil. Mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are suitable phosphates, such as salts of the phosphoric ester of a p-nonylphenol/(4-14)ethylene oxide adduct, or phospholipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl) phosphate, tris-n-octyl phosphate and tris-butoxyethyl phosphate, where tris-(2-ethylhexyl) phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-1 1 1-82-0), methyl palmitate (CAS-1 12-39-0) and methyl oleate (CAS-1 12-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037,373.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecyl-benzylsulfonate type, especially the calcium salts thereof, and also non-ionic surfactants of the fatty alcohol ethoxylate type. Special preference is given to ethoxylated C12-C22 fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Syngenta AG, CH) and Actipron® (BP Oil UK Limited, GB).

The said surface-active substances may also be used in the formulations alone, that is to say without oil additives. Furthermore, the addition of an organic solvent to the oil additive/surfactant mixture can contribute to a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE®(BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

As a rule, the compositions comprise 0.1 to 99%, especially 0.1 to 95%, of active ingredient of the compound described herein and 1 to 99.9%, especially 5 to 99.9%, of at least one solid or liquid adjuvant, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

Preferred compositions are composed in particular as follows (%=percent by weight):

Emulsifiable concentrates:
active ingredient 1 to 95%, preferably 5 to 50%, more preferably 5 to 20%
surfactant: 1 to 30%, preferably 10 to 20%
solvent: 5 to 98%, preferably 70 to 85%

Dusts:
active ingredient: 0.1 to 10%, preferably 2 to 5%,
solid carrier: 99.9 to 90%, preferably 99.9 to 99%

Suspension concentrates:
active ingredient: 5 to 75%, preferably 10 to 50%, more preferably 10 to 40%
water: 94 to 24%, preferably 88 to 30%
surfactant: 1 to 40%, preferably 2 to 30%

Oil-based suspension concentrates:
active ingredient: 2 to 75%, preferably 5 to 50%, more preferably 10 to 25%
oil: 94 to 24%, preferably 88 to 30%
surfactant: 1 to 40%, preferably 2 to 30%

Wettable powders:
active ingredient: 0.5 to 90%, preferably 1 to 80%, more preferably 25 to 75%
surfactant: 0.5 to 20%, preferably 1 to 15%
solid carrier: 5 to 99%, preferably 15 to 98%

Granulates:
active ingredient: 0.5 to 30%, preferably 3 to 25%, more preferably 3 to 15%
solid carrier: 99.5 to 70%, preferably 97 to 85%

Preferably, the term “active ingredient” refers to a compound of formula I, II, or III as described above. It also refers to mixtures of a compound of formula I, II, or III, in particular, a compound selected from said Tables 1 to 3, with other insecticides, fungicides, herbicides, safeners, adjuvants and the like, which mixtures are specifically disclosed below.

The compositions can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers; fertilizers, in particular nitrogen containing fertilizers such as ammonium nitrates and urea as described in WO08/017,388, which can enhance the efficacy of the inventive compounds; or other active ingredients for achieving specific effects, for example ammonium or phosphonium salts, in particular halides, (hydrogen)sulphates, nitrates, (hydrogen)carbonates, citrates, tartrates, formiates and acetates, as described in WO07/068,427 and WO07/068,428, which also can enhance the efficacy of the inventive compounds and which can be used in combination with penetration enhancers such as alkoxalated fatty acids; bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds for the preparation of these compositions are also a subject of the invention. The application methods for the compositions, such as spraying, atomizing, dusting, brushing on, dressing, scattering or pouring are to be selected to suit the intended aims of the prevailing circumstances. Typical rates of concentration are between 0.1 and 1000 ppm, preferably between 0.1 and 500 ppm, of active ingredient. The rate of application per hectare is generally 1 to 2000 g of active ingredient per hectare (ha), in particular 10 to 1000 g/ha, preferably 10 to 600 g/ha.

A preferred method of contacting plants in the field of crop protection is application to the foliage of the plants (foliar application), it being possible to select frequency and rate of application to match the severity of the conditions in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field. The compositions according to the invention are also suitable for the protection of plant propagation material, for example seeds, such as fruit, tubers or kernels, or nursery plants, against pests of the abovementioned type. The propagation material can be treated with the compositions prior to planting, for example seed can be treated prior to sowing.

Alternatively, the compositions can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed treatment formulations. The formulation type chosen in any instance will depend upon the particular purpose envisaged and the physical, chemical and biological properties of compound of the invention. Dustable powders (DP) may be prepared by mixing an active compound with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of the invention with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the invention and one or more powdered solid diluents or carriers, or from preformed blank granules by absorbing a compound of the invention (or a solution thereof, in a suitable agent) in a porous granular material (such as pumice, attapulgite clays, fuller's earth, kieselguhr, diatomaceous earths or ground corn cobs) or by adsorbing a compound of the invention (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent). Dispersible Concentrates (DC) may be prepared by dissolving a compound of the invention in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of the invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈₋₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of the invention either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the invention is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the invention. SCs may be prepared by ball or bead milling the solid compound of the invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the invention may be dry milled and added to water, containing agents hereinbefore described, to produce the desired end product.

Oil-based suspension concentrate (OD) may be prepared similarly by suspending finely divided insoluble solid particles of a compound of the invention in an organic fluid (for example at least one mineral oil or vegetable oil). ODs may further comprise at least one penetration promoter (for example an alcohol ethoxylate or a related compound), at least one non-ionic surfactants and/or at least one anionic surfactant, and optionally at least one additive from the group of emulsifiers, foam-inhibiting agents, preservatives, anti-oxidants, dyestuffs, and/or inert filler materials. An OD is intended and suitable for dilution with water before use to produce a spray solution with sufficient stability to allow spray application through appropriate equipment.

Aerosol formulations comprise a compound of the invention and a suitable propellant (for example n-butane). A compound of the invention may also be dissolved or dispersed in a suitable medium (for example water or a water miscible liquid, such as n-propanol) to provide compositions for use in non-pressurized, hand-actuated spray pumps.

A compound of the invention may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of the invention and, optionally, a carrier or diluent therefore. The polymeric shell may be produced by either an interfacial polycondensation reaction or by a coacervation procedure. The compositions may provide for controlled release of the compound of the invention and they may be used for seed treatment. A compound of the invention may also be formulated in a biodegradable polymeric matrix to provide a slow, controlled release of the compound. A compound of the invention may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC, OD and DC compositions described above.

Compositions for treating seed may include an agent for assisting the adhesion of the composition to the seed (for example a mineral oil or a film-forming barrier). A composition of the present invention may include one or more additives to improve the biological performance of the composition (for example by improving wetting, retention or distribution on surfaces; resistance to rain on treated surfaces; or uptake or mobility of a compound of the invention). Such additives include surface active agents (SFAs), spray additives based on oils, for example certain mineral oils, vegetable oils or natural plant oils (such as soy bean and rape seed oil), and blends of these with other bio-enhancing adjuvants (ingredients which may aid or modify the action of a compound of the invention). Increasing the effect of a compound of the invention may for example be achieved by adding ammonium and/or phosphonium salts, and/or optionally at least one penetration promoter such as fatty alcohol alkoxylates (for example rape oil methyl ester) or vegetable oil esters.

Wetting agents, dispersing agents and emulsifying agents may be surface active agents (SFAs) of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-/'sopropyl- and tri-/'sopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or define sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins. Suitable suspending agents include hydrophilic colloids (such as polysaccharides, polyvinylpyrrolidone or sodium carboxymethylcellulose) and swelling clays (such as bentonite or attapulgite).

Compositions for use as aqueous preparations (aqueous solutions or dispersions) are generally supplied in the form of a concentrate containing a high proportion of the active ingredient, the concentrate being added to water before use. These concentrates, which may include DCs, SCs, ODs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of the invention (for example 0.0001 to 10%, by weight) depending upon the purpose for which they are to be used.

A compound of the invention may be used in mixtures with fertilizers (for example nitrogen-, potassium- or phosphorus-containing fertilizers, and more particularly ammonium nitrate and/or urea fertilizers). Suitable formulation types include granules of fertilizer. The mixtures suitably contain up to 25% by weight of the compound of the invention.

In some embodiments, the invention therefore also provides a fertilizer composition comprising a fertilizer and a compound of the invention.

The compositions of this invention may contain other compounds having biological activity, for example micronutrients or compounds having fungicidal activity or which possess plant growth regulating, herbicidal, safening, insecticidal, nematicidal or acaricidal activity.

“Contacting the plant”, “applying to the plant” and “treating the plant” have the same meaning in the context of the present invention. Contacting the plants can be performed using a variety of known methods, e.g., by spraying, atomizing, dusting or scattering the compositions over the propagation material or brushing or pouring or otherwise contacting the compositions over the plant or, in the event of seed, by coating, encapsulating, or otherwise treating the seed. In an alternative to directly treating a plant or seed before planting, the formulations of the invention can also be introduced into the soil or other media into which the seed is to be planted. In some embodiments, a carrier is also used in this embodiment. The carrier can be solid or liquid, as noted above. In some embodiments peat is suspended in water as a carrier of the ABA agonist, and this mixture is sprayed into the soil or planting media and/or over the seed as it is planted.

In one aspect, the invention relates to a method of improving resistance to stress in a plant by contacting the plant with a compound of the invention, or a composition or formulation as described herein.

In one embodiment of this aspect, the plant is a land plant. In another embodiment, the land plant is a moss. In another embodiment, the land plant is a fern.

In another embodiment of this aspect, the plant is an underwater plant. In a further embodiment, the plant is green algae.

In one embodiment of this aspect, the compound, or composition or formulation comprising the compound, is applied directly to the plant.

In one embodiment, the compound, or composition or formulation comprising the compound, is applied directly to the seed of the plant.

In another embodiment, the compound, or composition or formulation comprising the compound, is applied directly to the soil in which the plant grows, or will grow.

In one embodiment, the stress is an abiotic stress. In another embodiment, the stress is a biotic stress. In a further embodiment, the stress is an abiotic stress selected from drought, high salinity, osmotic stress, heat or cold. In another further embodiment, the stress is a biotic stress from a microorganism. In another embodiment, the stress is drought. In still another embodiment, the stress is high salinity. In yet another embodiment, the stress is osmotic stress. In still another embodiment, the osmotic stress is high sugar concentration.

### Methods

Here we report the identification and mechanism of pyrabactin as a PYL2 selective antagonist. Crystal structures of the PYL2-pyrabactin antagonist complex and the PYL1-pyrabactin-ABI1 agonist complex reveal that pyrabactin adopts two distinct configurations, with a pseudo-symmetry that is flipped 180° between the agonist and antagonist conformation. In the PYL1 structure, pyrabactin mimics ABA and induces the gate closure of the receptor. In contrast, pyrabactin in the PYL2 structure prevents the closure of the ligand entry gate, therefore blocking the ability of PYL2 to bind and inhibit PP2C effectors. Using rational mutational design, we have converted PYL1 into a pyrabactin-inhibited receptor and PYL2 into a pyrabactin-activated receptor. Crystal structures of the altered-specificity PYL2 receptor bound to pyrabactin and in complexes with pyrabactin and the ABI2 and HAB1 PP2C illustrate the molecular basis of this conversion. Finally, we have explored the structural information for the identification of novel ABA receptor ligands. These results demonstrate a new concept of ABA receptor antagonism, lay a theoretic foundation for future identification of physiological ABA receptor antagonists, and establish a structural framework to screen and design subtype-selective agonists and antagonists for unraveling ABA biology.

Pyrabactin was identified as a selective agonist of PYR1 in a seed germination assay²¹. It also promoted interaction of the HAB1 PP2C with PYR1, PYL1, and PYL3 but not with PYL2 and PYL4 in yeast two-hybrid assays¹. To determine the biochemical basis of these observations, we used purified proteins of PYR1 and PYL1 to PYL6 to measure their interactions with three PP2Cs, HAB1, ABI1, and ABI2, in vitro. Pyrabactin strongly promoted PYR1 to interact with all three PP2Cs, consistent with its original identification as a selective PYR1 agonist. Pyrabactin also promoted interaction of PYL1, PYL3, PYL6, and surprisingly PYL4 with the three PP2Cs. In contrast, pyrabactin did not promote PYL2 interaction with any of the three PP2Cs. In phosphatase assays, pyrabactin promoted PYR1, PYL1, PYL3, PYL5, and PYL6, but not PYL2 and PYL4, to inhibit all three PP2Cs. Interestingly, in contrast to other receptors analyzed, PYL6 has high basal activity in both PP2C binding and inhibition. These results demonstrate different modes of PP2C binding and PP2C inhibition for different members of the PYR/PYL family.

Surprisingly, not only did pyrabactin fail to activate PYL2, but it also inhibited ABA-dependent PYL2 interaction with all three PP2Cs in a concentration-dependent manner, suggesting pyrabactin may be a PYL2 selective antagonist. Consistent with this observation, pyrabactin did not promote PYL2 to inhibit the three PP2Cs, but high concentrations of pyrabactin reversed ABA-dependent inhibition of PP2Cs by PYL2. In addition, pyrabactin promoted PYR1 in a concentration-dependent manner to induce the expression of RD29B-LUG, an ABA-inducible reporter, but inhibited PYL2 and ABA-dependent induction in a reconstituted ABA signaling pathway in Arabidopsis protoplasts²². These results collectively establish that pyrabactin is a PYL2-selective antagonist.

To understand the molecular basis of pyrabactin as a subtype-selective agonist and antagonist of ABA receptors, we determined the crystal structures of a PYL1-pyrabactin-ABI1 ternary complex and a PYL2-pyrabactin binary complex at resolutions of 2.15 Å and 1.85 Å, respectively. These structures were solved by molecular replacement starting from the PYL1-ABA-ABI1 and apo-PYL2 structures^{13,14,17}, with the statistics of data and structures summarized in Table 4. For the PYL1-ABA-ABI1 complex, there are two ternary complexes in each asymmetric unit. Consistent with the agonist property of pyrabactin for PYL1, the overall arrangement of the PYL1-pyrabactin-ABI1 complex resembles the agonist structure of the PYL1-ABA-ABI1 complex, with the gate and latch loops of PYL1 (residues 112-116 and 142-144) adopting the closed conformation that is further stabilized by the insertion of the locking residue, W300 from ABI1. In the complex, the gate/latch interface is tightly packed against the active site of ABI1, therefore providing a mechanism of phosphatase inhibition.

Pyrabactin, which is clearly defined by a high resolution electron density map, adopts a π-shape configuration in the PYL1 pocket. The binding mode of pyrabactin mimics that of ABA with the naphthalene double ring of pyrabactin overlapping extensively with the cyclohexene ring of ABA. The bromide group from the naphthalene ring forms several Van der Waals interactions with LPA, three residues from the ligand entry gate, and these interactions are important to keep the gate in the closed conformation as the equivalent P88S mutation in PYR1 abolishes its responses to pyrabactin¹. The nitrogen from the pyridine ring of pyrabactin functionally mimics the acidic group of ABA and forms a water-mediated hydrogen bond with K86. These interactions between pyrabactin and PYL1 provide a basis for understanding the structure-functional relationship of pyrabactin derivatives, and help to explain why the nitrogen group of pyridine is required for agonist activity of pyrabactin¹.

The PYL2-pyrabactin crystals were obtained under the same conditions as for the ABA-bound PYL2, but were formed in the same space group as the apo-PYL2. In contrast to the closed conformation of PYL1, pyrabactin-bound PYL2 adopts an open conformation where its ligand entry gate assumes a position similar to the apo-PYL2 structure. There are three PYL2-pyrabactin complexes in each asymmetric unit and pyrabactin assumes a similar π configuration in each complex. Interestingly, the binding orientation of pyrabactin in PYL2 is flipped 180° from that of PYL1. In this conformation, pyrabactin forms extensive interactions with PYL2. The sulfonamide functional group mimics the acidic group of ABA, forming two water-mediated hydrogen bonds with K64 (K89 equivalent in PYL1) and one water mediated hydrogen bond with E98. The naphthalene ring of pyrabactin forms parallel packing interactions with the phenol ring of Y124 and the pyridine ring of pyrabactin. We also determined the crystal structure of pyrabactin itself, which adopts a more extended conformation than that in the ligand binding pockets of PYL1 and PYL2, indicating that pyrabactin undergoes an induced fit to accommodate the shape of the ligand binding pockets.

One key question is why pyrabactin adopts different conformations between PYL1 and PYL2. The ligand binding pockets of PYL2 and PYL1 only differ in three residues (PYL2/PYL1: V114/1137, V166/A190, and V170/1194). Structure modeling indicated that V166/A190 and V170/1194 changes would not interfere with the binding of pyrabactin in either the PYL1 or the PYL2 conformation. In contrast, the V114/1137 change in PYL1 collides with the naphthalene ring of pyrabactin in the PYL2 conformation (C—C distance of 2.2 Å) and forces pyrabactin to flip by 180°. Mutation of PYL1 1137 to valine, which was predicted to allow pyrabactin to adopt the PYL2-bound conformation, converted PYL1 to a pyrabactin-inhibited receptor. Due to the very weak pyrabactin agonist activity in wildtype PYL1, this change is most clearly seen in the context of two mutant PYL1 receptors, in which residues A190 and V193 from helix 3 were exchanged against larger side chains that stabilize the binding of pyrabactin. A190V and V193I mutations strongly increased PYL1 agonist activity. Introduction of the I137V mutation into either PYL1 A190V or PYL1 V193I strongly shifted the equilibrium from a predominantly pyrabactin-activated receptor to a predominantly pyrabactin-antagonized receptor, for which pyrabactin inhibited ABA-promoted activation in a concentration-dependent manner. From these results we conclude that removal of a single methyl group (isoleucine vs. valine) is sufficient to convert PYL1 from pyrabactin-activated receptor into a pyrabactin-repressed receptor.

Another key question is why pyrabactin binding does not activate PYL2. Structural analysis indicates that pyrabactin in the context of PYL2 does not form any direct interactions with residues from the ligand entry gate. The closest distance between the pyridine ring of pyrabactin and the LPA gating residues is 4.8 Å-7.0 Å when the LPA loop assumes the ABA-bound closed conformation, but is 11-13 Å when the LPA gate is in open conformation. In contrast, the distance of the naphthalene double ring of pyrabactin or the cyclohexene ring of ABA to the three side chains of the LPA gate in the PYL1 structure is 3.4-4.6 Å, a range for strong Van der Waals interactions, thus helping to keep the LPA gate in the closed conformation. These observations suggest that the antagonism of pyrabactin to PYL2 is because the pyridine group is positioned too far away to make any direct contacts with the LPA loop that would help keep the gate closed. To validate this hypothesis, we changed the alanine in the LPA loop to a larger residue. Indeed, mutation of A93F, designed to close the distance between the gate loop and the pyridine ring, converted PYL2 from a pyrabactin-repressed to a pyrabactin-activated receptor, as determined by PP2C binding and phosphatase assays. As expected from structural modeling, replacement of the gate residue A93 with the bulky phenylalanine ring also abrogated ABA to promote PYL2 activation.

To gain further understanding on how the A93F mutation converts PYL2 into a pyrabactin-activated, ABA-insensitive receptor, we solved the crystal structures of a dimeric complex of PYL2 A93F bound to pyrabactin at a resolutions of 2.10 Å and two trimeric complexes of PYL2 A93F-pyrabactin bound to ABI2 and HAB1 at a resolution of 2.10 Å and 2.55 Å, respectively. The two trimeric structures closely resemble the active PYL2-ABA-HAB1¹³ and PYL1-ABA-ABI1^{14,17} structures, with the gate and latch loops in the closed conformation, and the PP2C locking residue (W290 in ABI2 and W385 in HAB1) inserted between the gate and latch to make water-mediated contacts with the bound pyrabactin. Pyrabactin adopts an intermediate conformation between the agonist and antagonist conformation to allow formation of Van der Waals interactions with F93, which contacts both ring systems of pyrabactin, thereby stabilizing the gate in the closed conformation as predicted. The ABI2 structure, which had not been solved previously, resembles the PP2C structure of ABI1 and HAB1^{13,14,17}.

The dimeric PYL2 A93F-pyrabactin structure shows mixed conformations of the activated and repressed receptors for the ligand entry gate and latch loops. Although the gate adopts an open conformation with F93 facing away from the ligand binding pocket as A93 in wildtype apo PYL2, the latch is deposited into the active conformation. The latch residue H119 is flipped into the ligand binding pocket mimicking its conformation in the active trimeric complex and forms direct contacts with pyrabactin. E118, the residue immediately preceding the HRL latch, is flipped outside of the pocket, as in ABA-activated PYL2¹³, to allow closure of the gate onto the latch upon PP2C binding. In this state, the mutated receptor is poised to be activated upon the binding of pyrabactin and a PP2C, therefore providing the basis of pyrabactin agonism by the mutated receptor.

The ability to switch the activation/repression response of PYL1 and PYL2 to pyrabactin by single point mutations demonstrates the detailed levels of our mechanistic understanding of ABA receptor activation and repression, and provides a rational model to screen for novel ABA receptor activators and inhibitors. To demonstrate the validity of this approach, we explored the structural information of pyrabactin bound to PYL1 and PYL2. In both the PYL1 and PYL2 structures, the sulfonamide group of pyrabactin forms extensive hydrogen bond interactions with the conserved pocket residues. We therefore searched virtual library servers representing more than 10 million commercially available compounds for molecules that contain the sulfonamide group attached to a naphtalene frame. These compounds were then computationally docked into the PYL1 ligand binding pocket. To validate our docking approach, we tested 64 of the top 100 docking matches for their ability to promote PYR1-PP2C interaction and PYR1-dependent inhibition of PP2C activity in vitro. Some of the 64 compounds efficiently activated PYR1 with efficacies and affinities similar to that of pyrabactin. The success of this virtual docking exercise demonstrates the proof of concept for future screening and design of potent ABA receptor ligands to explore ABA biology and for agriculture applications.

Here, we present comprehensive evidence for the phenomenon of ABA receptor antagonism and its underlying mechanisms through combinatorial approaches of structural, biochemical, mutagenesis and chemical screening studies. The antagonism concept of ABA receptors mirrors the mechanism of the receptor activation and has profound implications in the regulation of ABA receptor physiology. It is well documented that the concentration of endogenous ABA in unstressed plant tissues is in a range that is sufficient to bind and activate several different recombinant PYL/PP2C complexes^{1-3,13,18,23,24}. In addition, some ABA receptors, including PYL6, have high level of constitutive activity in PP2C inhibition, and yet the ABA response is silent in normal unstressed plants. This raises the important question of how the basal activity of ABA receptors is inhibited. Our data lead to the intriguing hypothesis of the existence of physiological antagonists that can inhibit the basal activation of ABA receptors. We speculate that likely candidates are catabolic derivatives or storage forms of ABA, as ABA metabolic products are known to be involved in ABA responses²⁴. Validation of this hypothesis by identifying the endogenous ABA receptor antagonists will be an exciting direction for future research.

The mechanisms of ABA receptor antagonism presented here also complement the activation mechanism of ABA receptors elucidated by earlier structural studies¹³⁻¹⁷, and provide a full picture for up and down regulation of ABA receptors. This detailed mechanistic understanding of the receptor regulation has allowed us to manipulate the receptor activation and repression properties as well as ligand specificity, which will be important new tools for metabolic engineering and to unravel the biology of individual receptors in the context of high receptor redundancy. Furthermore, the structure information of the ABA receptor agonism and antagonism provides a solid framework for computational screening of virtual chemical libraries, which have allowed us to identify novel ABA receptor activators. ABA signaling plays the central role in plant resistance to environmental stresses such as drought conditions. The ability of identifying agonists of ABA receptors opens a new avenue for making other small molecules of ABA mimics, which should have practical applications for improving crop yield under stress conditions.

### EXAMPLES

General Methods

PYL2, PYL1, ABI1, ABI2, and HAB1 were expressed as H6GST or H6Sumo fusion proteins in E. coli. Proteins were purified by Ni-NTA chromatography, followed by proteolytic release of tags and size-exclusion chromatography. For formation of PYL2-pyrabactin, ABI1-PYL1-pyrabactin, ABI2-PYL2 A93F-pyrabactin, and HAB1-PYL2 A93F-pyrabactin complexes, pyrabactin was mixed with PYL2, PYL1-ABI1, and PYL2 A93F-PP2C complexes at 5:1 ratios. Crystals were grown by vapor diffusion and diffraction data were collected from cryo-protected crystals at beamlines 21-ID-D and 21-ID-F at the Advanced Photon Source at Argonne National Laboratories. Structures were solved by molecular replacement in PHASER²⁵ using the structures of PYL1/ABA/ABI1 and apo-PYL2 as models for PYL1/pyrabactin/ABI1 and PYL2-pyrabactin, respectively. Models were manually fitted using O and Coot^{26,27} and further refined using CNS and Refmac5^{28,29}.

Mutant proteins were expressed as H6GST-fusion proteins and purified by glutathione sepharose chromatography. Protein-protein interactions were determined by luminescence proximity AlphaScreen assay. Biotinylated PP2C proteins for the luminescence proximity assay were generated by in vivo biotinylation of avitag-PP2C fusion proteins. PP2C phosphatase activity was measured by phosphate release from a SnRK2.6 phosphoprotein.

The ABA signal transduction pathway was reconstituted in protoplasts by transient transfection of PYR/PYL receptor, ABI1, SnRK2.6, and ABF2 expression plasmids. Activation of an ABA-inducible RD29B promoter-LUC reporter was determined by luciferase assays normalized for β-glucuronidase activity from a UQ10-GUS reporter.

### Example 1

### Protein Preparation

PYL1 (residues 36-211), PYL2 (residues 14-188), and HAB1 (residues 172-511) were prepared as recombinant proteins in E. coli as described¹³. ABI1 (residues 117-434) and ABI2 (residues 101-423) were expressed as H6Sumo fusion proteins from the expression vector pSUMO (LifeSensors). Expression in E. coli BL21 and purification followed the same general method as for PYL1¹³. To prepare PYL-pyrabactin complexes, we incubated purified PYL1 and PYL2 with pyrabactin at a 1:5 molar ratio for 30 minutes on ice prior to crystallization trials. For ternary complexes, we added pyrabactin and purified PYL proteins to purified PP2Cs at a 5:1:1 molar ratio in the presence of 5 mM MgCl₂.

Small scale purifications of H6GST-tagged PYL/PYR proteins for binding studies of wildtype and mutant proteins were performed by standard glutathione sepharose chromatography. Biotinylated PP2C proteins were prepared as recombinant fusion proteins with in vivo biotinylated 14 amino acid avitags as previously described for HAB1¹³.

Crystallization

The PYL2-pyrabactin crystals were grown at room temperature in hanging drops containing 2.4 μl of the purified PYL2 protein at 16.8 mg/ml and 1.6 μl of well solution containing 2M ammonium sulfate, 0.1M HEPES pH 7.5 and 10% glycerol. Crystals appeared within 1-2 days and grew to a dimension of approximately 250 μm in length on the 6th day. Crystals were transferred to well buffer with 20% v/v glycerol prior to flash freezing in liquid nitrogen.

The PYL1-pyrabactin-ABI1 complex crystals were grown at room temperature in hanging drops containing 1.0 μl of the above protein-ligand-PP2C solutions and 1.0 μl of well solution containing 0.2M ammonium sulphate, 0.1M BisTris pH 5,5, and 22% PEG 3350. Crystals appeared within 1-2 days and grew to a dimension of about 120 μm in length on the 4th day. Crystals were serially transferred to well buffer with increasing PEG3350 concentration (35% v/v final) before flash freezing in liquid nitrogen.

The PYL2 A93F-pyrabactin crystals were grown at room temperature in hanging drops containing 1.0 μl of the purified PYL2 protein at 13.1 mg/ml and 1.0 μl of well solution containing 2M ammonium acetate pH 8.1 and 22% PEG 3350. Crystals appeared within 1-2 days and grew to a dimension of about 150-200 μm in length after 2 weeks. Crystals were serially transferred to well buffer with increasing PEG 3350 concentration (40% v/v final) prior to flash freezing in liquid nitrogen.

The PYL2 A93F-pyrabactin-ABI2 complex crystals were grown at 4° C. in hanging drops containing 1.6 μl of the protein-ligand-PP2C solutions and 2.4 μl of well solution containing 0.1M HEPES pH 7.5, 10% PEG 8000 and 10% sucrose. Crystals appeared within 1-2 days and grew to a dimension of about 200 μm in length on the 4th day. Crystals were serially transferred to well buffer with increasing sucrose concentration (35% final) before flash freezing in liquid nitrogen.

The PYL2 A93F-pyrabactin-HAB1 complex crystals were grown at room temperature in hanging drops containing 1.0 μl of the protein-ligand-PP2C solution and 1.0 μl of well solution containing 0.2M ammonium sulphate, 0.1M Tris pH 7.5, 10% ethyl glycol and 23% PEG 3350. Crystals were grown to a dimension of about 250 μm in length and flash-frozen in liquid nitrogen on the 3^{rd} day.

Pyrabactin crystals were grown at room temperature in hanging drops containing 1 μl of 40 mM pyrabactin in methanol and 1 μl of well solution containing 50% 2-methyl-2,4-pentanediol. Crystals were grown to a dimension of 100 μm in length and flash-frozen in liquid nitrogen on the 2^{nd} day.

Data Collection and Structure Determination

The diffraction data was collected with MAR300 and MAR225CCD detectors (MAR Research) at the ID-D and ID-F beamlines of sector-21 (LS-CAT) at the Advanced Photon Source at Argonne National Laboratory. The observed reflections were reduced, merged, and scaled with DENZO and SCALEPACK in the HKL2000 package³⁰. Crystals of PYL1-pyrabactin-ABI1, PYL2-pyrabactin, free pyrabactin, PYL2 A93F-pyrabactin, PYL2 A93F-pyrabactin-ABI2, and PYL2 A93F-pyrabactin-HAB1 diffracted to resolutions of 2.15 Å, 1.85 Å, 0.70 Å, 2.10 Å, 2.10 Å and 2.55 Å, respectively.

Crystals of PYL1-pyrabactin-ABI1 formed in the P1 space group with two receptor/PP2C complexes in each unit cell. Both crystals of PYL2-pyrabactin and PYL2 A93F-pyrabactin complexes belonged to the C222₁ space group and had similar unit cell parameters, with three complex monomers in each asymmetric unit. The crystals of PYL2 A93F-pyrabactin-ABI2 and PYL2 A93F-pyrabactin-HAB1 formed in the P2₁2₁2₁ space group with different cell dimensions, and both asymmetric units contained one complex monomer. The crystal of free pyrabactin belonged to the space group of P2₁/c with 4 molecules per unit cell.

Molecular replacement was performed by using the Collaborative Computational Project 4 (CCP4) program Phaser²⁵. Programs O and Coot were used to manually fit the protein model^{26,27}. Model refinement was performed with CNS and the CCP4 program Refmac5^{28,29}. The volumes of the ligand binding pocket were calculated with the program Voidoo by using program default parameters and a probe with a radius of 1.4 Å³¹. All structure figures were prepared by using PyMOL (DeLano Scientific). The statistics of data collection and the model refinement are summarized in Table 4.

TABLE 4
Data collection and structure determination statistics
Table 4: Data collection and structure determination statistics

| | | PYL1- | | PYL2 A93F- | PYL2 A93F- |
|---|---|---|---|---|---|
| | PYL2- | pyrabactin- | PYL2 A93F- | pyrabactin- | pyrabactin- |
| | pyrabactin | ABI1 | pyrabactin | HAB1 | ABI2 |
| Data collection | | | | | |
| APS beam line | 21-ID | 21-ID | 21-ID | 21-ID | 21-ID |
| Space group | C222₁ | P1 | C222₁ | P2₁2₁2₁ | P2₁2₁2₁ |
| Resolution, Å | 30-1.85 | 30-2.15 | 30-2.10 | 30-2.55 | 30-2.10 |
| Cell parameters, Å, ° | a = 62.27, | a = 59.98, | a = 62.08, | a = 61.02, | a = 62.13, |
| | b = 105.07, | b = 66.71, | b = 105.57, | b = 66.88, | b = 97.59, |
| | c = 185.08; | c = 72.60; | c = 182.90; | c = 144.44; | c = 134.50; |
| | α = β = γ = 900 | α = 115.8, | α = β = γ = 90 | α = β = γ = 90 | α = β = γ = 90 |
| | | β = 95.4; | | | |
| | | γ = 105.6 | | | |
| Total/Unique | 646811/51071 | 140047/50236 | 291389/35374 | 164481/19661 | 511848/41807 |
| reflections | | | | | |
| Completeness, % | 96.9 (92.8) | 97.6 (96.2) | 99.9 (99.9) | 100.0 (100.0) | 100.0 (100.0) |
| I/σ | 32.2 (3.9) | 49.1 (4.5) | 27.6 (2.9) | 15.9 (2.4) | 32.4 (3.7) |
| Redundancy | 12.7 (12.8) | 13.4 (2.3) | 8.2 (8.4) | 8.4 (8.6) | 14.7 (14.6) |
| Rsym | 0.10 (0.706) | 0.098 (0.752) | 0.093 (0.725) | 0.164 (0.793) | 0.102 (0.764) |
| Structure determination | | | | | |
| Resolution, Å | 30-1.85 | 30-2.15 | 30-2.10 | 30-2.55 | 30-2.10 |
| No. reflections | 18156 | 46283 | 32811 | 18181 | 25445 |
| No. residues | 528 | 888 | 528 | 472 | 456 |
| No. solvent molecules | 354 | 101 | 273 | 137 | 172 |
| No. of non-H atoms | 4643 | 7243 | 4511 | 3877 | 3599 |
| Rcryst | 19.1% | 21.2% | 23.0% | 22.3% | 19.4% |
| Rfree | 23.6% | 25.5% | 26.9% | 26.0% | 22.3% |
| Rmsd^{‡} bonds, Å | 0.018 | 0.021 | 0.022 | 0.018 | 0.023 |
| Rmsd^{‡} angles, ° | 1.71 | 1.73 | 1.66 | 1.44 | 1.74 |
| Average B factor, Å² | 32.07 | 41.41 | 42.63 | 41.62 | 36.0 |

^{‡}Rmsd is the root-mean-square deviation from ideal geometry of protein.

Molecular Modeling

Starting with the pyrabactin structure, substructure searching was performed using Canvas (version 1.2, Schrödinger, LLC, New York) on Chembridge (800,000 screening compounds) and ZINC8 (13 million compounds) databases. Identified compounds were further subjected to cluster analysis by using ICM/Molsoft (version 3.5-1n, Molsoft LLC) and molecular docking using Glide/Schrodinger (version 5.5, Schrödinger, LLC, New York). 64 of the top scoring 100 compounds were purchased from Chembridge, Otava, and Princeton BioMolecular Research. Alternatively, the compounds of the invention can be synthesized according to methods known or available to those having skill in the art.

### Example 2

### Assays for the Interactions Between PYR/PRL and PP2Cs

Interactions between PYR/PYL and PP2Cs were assessed by luminescence-proximity AlphaScreen technology (Perkin Elmer). Briefly, biotinylated PP2Cs were attached to streptavidin-coated donor beads, and H6-tagged PYR/PYL receptors were attached to nickel-chelated acceptor beads. The donor and acceptor beads were brought into proximity by the interactions between PYR/PYLs and PP2Cs, which were measured with and without ligands. When excited by a laser beam of 680 nm, the donor beam emits singlet oxygen that activates thioxene derivatives in the acceptor beads, which releases photons of 520-620 nm as the binding signal. The experiments were conducted with 100 nM of PP2Cs and PYR/PYL proteins in the presence of 5 ug/ml donor and acceptor beads in a buffer of 50 mM MOPS, pH7.4, 50 mM NaF, 50 mM CHAPS, and 0.1 mg/ml bovine serum albumin. The results were based on an average of three experiments with standard errors typically less than 10% of the measurements.

Table 5a shows the stimulation of the interaction between 100 nM His6-PYR/PYL receptor and 100 nM biotin-PP2C in the presence or absence of 100 μM agonist determined by AlphaScreen luminescence proximity assay. Values were determined in different experiments and normalized against a pyrabactin control. Blank spaces mean no experimental data is available. Key (photon counts (binding signal units) in Alphascreen luminescence proximity assay): “+++ is >75,000; ++ is 25,000−75,000; + is <25,000.

TABLE 5a
Stimulation of the interaction between PYR1
and PP2Cs by the compounds of the invention:

| Compound # | PYR1 | PYL1 | PYL2 | PYL3 |
|---|---|---|---|---|
| 1 | + | + | + | + |
| 2 | +++ | + | + | + |
| 3 | ++ | | + | +++ |
| 4 | +++ | | | + |
| 5 | + | + | ++ | + |
| 6 | ++ | | + | ++ |
| 7 | ++ | ++ | ++ | + |
| 8 | + | | + | ++ |
| 9 | + | | + | + |
| 10 | + | | + | + |
| 11 | +++ | + | + | ++ |
| 12 | +++ | | | +++ |
| 13 | + | | | |
| 14 | + | | + | + |
| 15 | + | + | ++ | ++ |

The results shown in Table 5b were calculated from the dose response curves of the dependence of the interaction between 100 nM His6-PYR1 and 100 nM biotin-PP2C on the presence of 0-100 μM agonists determined by AlphaScreen luminescence proximity assay. Key (EC₅₀ in μM): ++++=<5.0 μM; +++=5-10 μM; ++=10-20 μM; and +=>20-60 μM.

TABLE 5b
EC₅₀ Measurements for the Inhibition of PP2C enzymes.

| Compound # | ABI1: EC₅₀ | ABI2: EC₅₀ | HAB1: EC₅₀ |
|---|---|---|---|
| Pyrabactin | +++ | +++ | ++++ |
| 1 | ++ | + | + |
| 2 | ++ | + | +++ |
| 3 | ++++ | ++++ | ++++ |
| 11 | ++ | + | +++ |

### Example 3

### Assays of PP2C Phosphatase Activity

PP2Cs and PYR/PYL proteins were pre-incubated at the indicated concentrations in 50 mM imidazole, pH 7.2, 5 mM MgCl₂, 0.1% β-mercaptoethanol and 0.5 mg/ml BSA for 30 min at room temperature. Reactions were started by addition of 100 μM of a phosphopeptide corresponding to amino acids 170-180 of SnRK2.6 (HSQPK^{p}STVGTP). This peptide is phosphorylated at a single residue corresponding to Ser175 in SnRK2.6, whose phosphorylation is required for SnRK2.6 kinase activity³². Phosphate release from ^{p}S175 from the phosphopeptide was determined by colorimetric assay (BioVision).

TABLE 6
Agonist-dependent inhibition of phosphatase activity of 200
nM PP2C incubated with 400 nM of a phosphopeptide corresponding
to amino acids 170-180 of SnRK2.6 (HSQPK^{p}STVGTP).

| Compound | % Phosphatase Activity | | |
|---|---|---|---|
| (μM) | ABI1 | ABI2 | HAB1 |
| None | 100 ± 3.3 | 100 ± 1.6 | 100 ± 1.9 |
| Pyrabactin | 2.8 ± 0.2 | 1.5 ± 0.8 | 12.6 ± 0.3 |
| 1 | 3.3 ± 1.0 | 3.2 ± 0.7 | 16.6 ± 2.0 |
| 2 | 4.3 ± 0.8 | 3.8 ± 0.6 | 6.1 ± 1.9 |
| 3 | 7.7 ± 4.3 | 7.0 ± 0.3 | 17.5 ± 0.6 |
| 11 | 4.7 ± 1.0 | 4.5 ± 0.5 | 8.8 ± 0.3 |

### Example 4

### Mutagenesis

Site-directed mutagenesis was carried out using the QuickChange method (Stratagene). Mutations and all plasmid constructs were confirmed by sequencing.

### Example 5

### Protoplast Transient Assays

An ABA-inducible luciferase reporter was constructed by replacing the CBF3 promoter in the CBF3-LUC construct with the promoter region of RD29B, a highly ABA-inducible gene². CBF3-LUC, UQ10-GUS and ABI1 protoplast expression plasmid vectors were kindly provided by Jen Sheen (Massachusetts General Hospital, Boston, Mass. 02114).

Protoplasts were isolated from Arabidopsis thaliana ecotype Columbia-0 plants, which were grown on Jiffy7 soil (Jiffy Products Ltd., Canada) in an environment-controlled chamber at 22° C. with a photosynthetically active radiation of 75 μmol m⁻² s⁻¹ and a day/night cycle of 13 hours light/11 dark. Transient activity assays were performed in Arabidopsis mesophyll protoplasts from Columbia-0 wild-type plants as described by the Sheen laboratory³³. Transfected protoplasts were incubated for 5 hours in light in the presence of 0 or 5 μM (+) ABA and the indicated concentrations of pyrabactin, and then used for measuring LUC and GUS activity. The UQ10-GUS reporter plasmid (β-glucuronidase) was used as internal control to normalize transfection efficiency in protoplast assays.

### Example 6

### Seed Germination Assays

Surface-sterilized Arabidopsis thaliana seeds were placed on 0.5× Murashige&Skoog agar plates and incubated at room temperature in daylight for 4 days. Twenty-four separate plates were incubated, each in the presence of 0 μM, 32 μM or 64 μM test compound. Table 7 below shows the activity of each compound on seed germination (i.e. during times of increased stress resistance, the seed will delay germination). Key (% growth relative to germination without test compound): +=>75%; ++=25%-75%; and +++=<25%.

TABLE 7
Inhibition of Germination by ABA agonist
compounds of the invention.

| Test Compound | | |
|---|---|---|
| (ABA agonist) | 32 μM Agonist | 64 μM Agonist |
| (+)-Abscisic Acid | +++ | +++ |
| Pyrabactin | ++ | +++ |
| 1 | + | + |
| 2 | ++ | ++ |
| 3 | ++ | ++ |
| 4 | ++ | +++ |
| 11 | +++ | +++ |
| 12 | + | + |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

### REFERENCES

¹ S. Y. Park, P. Fung, N. Nishimura et al. Abscisic acid inhibits type 2C protein phosphatases via the PYR/PYL family of START proteins. Science 324 (5930), 1068 (2009).
² Y. Ma, I. Szostkiewicz, A. Korte et al. Regulators of PP2C phosphatase activity function as abscisic acid sensors. Science 324 (5930), 1064 (2009).
³ J. Santiago, A. Rodrigues, A. Saez et al. Modulation of drought resistance by the abscisic acid receptor PYL5 through inhibition of Glade A PP2Cs. Plant J (2009).
⁴ T. Furihata, K. Maruyama, Y. Fujita et al. Abscisic acid-dependent multisite phosphorylation regulates the activity of a transcription activator AREB1. Proc Natl Acad Sci USA 103 (6), 1988 (2006).
⁵ Y. Kobayashi, M. Murata, H. Minami et al. Abscisic acid-activated SNRK2 protein kinases function in the gene-regulation pathway of ABA signal transduction by phosphorylating ABA response element-binding factors. Plant J 44 (6), 939 (2005).
⁶ N. Nishimura, T. Yoshida, N. Kitahata et al. ABA-Hypersensitive Germination1 encodes a protein phosphatase 2C, an essential component of abscisic acid signaling in Arabidopsis seed. Plant J 50 (6), 935 (2007).
⁷ C. Viswanathan and J. K. Zhu. Molecular genetic analysis of cold-regulated gene transcription. Philos Trans R Soc Lond B Biol Sci 357 (1423), 877 (2002).
⁸ L. Xiong, K. S. Schumaker, and J. K. Zhu. Cell signaling during cold, drought, and salt stress. Plant Cell 14 Suppl, S165 (2002).
⁹ K. Yamaguchi-Shinozaki and K. Shinozaki. Transcriptional regulatory networks in cellular responses and tolerance to dehydration and cold stresses. Annu Rev Plant Biol 57, 781 (2006).
¹⁰ R. Yoshida, T. Umezawa, T. Mizoguchi et al. The regulatory domain of SRK2E/OST1/SnRK2.6 interacts with ABI1 and integrates abscisic acid (ABA) and osmotic stress signals controlling stomatal closure in Arabidopsis. J Biol Chem 281 (8), 5310 (2006).
¹¹ T. Yoshida, N. Nishimura, N. Kitahata et al. ABA-hypersensitive germination3 encodes a protein phosphatase 2C (AtPP2CA) that strongly regulates abscisic acid signaling during germination among Arabidopsis protein phosphatase 2Cs. Plant Physiol 140 (1), 115 (2006).
¹² J. K. Zhu. Salt and drought stress signal transduction in plants. Annu Rev Plant Biol 53, 247 (2002).
¹³ K. Melcher, L. M. Ng, X. E. Zhou et al. A gate-latch-lock mechanism for hormone signalling by abscisic acid receptors. Nature 462 (7273), 602 (2009).
¹⁴ K. Miyazono, T. Miyakawa, Y. Sawano et al. Structural basis of abscisic acid signalling. Nature 462 (7273), 609 (2009).
¹⁵ N. Nishimura, K. Hitomi, A. S. Arvai et al. Structural mechanism of abscisic acid binding and signaling by dimeric PYR1. Science 326 (5958), 1373 (2009).
¹⁶ J. Santiago, F. Dupeux, A. Round et al. The abscisic acid receptor PYR1 in complex with abscisic acid. Nature 462 (7273), 665 (2009).
¹⁷ P. Yin, H. Fan, Q. Hao et al. Structural insights into the mechanism of abscisic acid signaling by PYL proteins. Nat Struct Mol Biol 16 (12), 1230 (2009).
¹⁸ P. McCourt and R. Creelman. The ABA receptors—we report you decide. Curr Opin Plant Biol 11 (5), 474 (2008).
¹⁹ M. J. Harris, W. H. Outlaw, R. Mertens et al. Water-stress-induced changes in the abscisic acid content of guard cells and other cells of Vicia faba L. leaves as determined by enzyme-amplified immunoassay. Proc Natl Acad Sci USA 85 (8), 2584 (1988).
²⁰ S. Q. Zhang, W. H. Outlaw, Jr., and K. Aghoram. Relationship between changes in the guard cell abscisic-acid content and other stress-related physiological parameters in intact plants. J Exp Bot 52 (355), 301 (2001).
²¹ Y. Zhao, T. F. Chow, R. S. Puckrin et al. Chemical genetic interrogation of natural variation uncovers a molecule that is glycoactivated. Nat Chem Biol 3 (11), 716 (2007).
²² H. Fujii, V. Chinnusamy, A. Rodrigues et al. In vitro reconstitution of an abscisic acid signalling pathway. Nature 462 (7273), 660 (2009).
²³ R Mertens, B Deus-Neumann, and E W Weiler. Monoclonal antibodies for the detection and quantitation of the endogenous plant growth regulator, abscisic acid. FEBS Lett 160, 269 (1983).
²⁴ D. Huang, W. Wu, S. R. Abrams et al. The relationship of drought-related gene expression in Arabidopsis thaliana to hormonal and environmental factors. J Exp Bot 59 (11), 2991 (2008).
²⁵ A. J. McCoy, R. W. Grosse-Kunstleve, P. D. Adams et al. Phaser crystallographic software. J Appl Crystallogr 40 (Pt 4), 658 (2007).
²⁶ P. Emsley and K. Cowtan. Coot: model-building tools for molecular graphics. Acta Crystallogr D Biol Crystallogr 60 (Pt 12 Pt 1), 2126 (2004).
²⁷ G. J. Kleywegt and T. A. Jones. Efficient rebuilding of protein structures. Acta Crystallogr D Biol Crystallogr 52 (Pt 4), 829 (1996).
²⁸ A. T. Brunger, P. D. Adams, G. M. Clore et al. Crystallography & NMR system: A new software suite for macromolecular structure determination. Acta Crystallogr D Biol Crystallogr 54 (Pt 5), 905 (1998).
²⁹ G. N. Murshudov, A. A. Vagin, A. Lebedev et al. Efficient anisotropic refinement of macromolecular structures using FFT. Acta Crystallogr D Biol Crystallogr 55 (Pt 1), 247 (1999).
³⁰ Z. Otwinowski, D. Borek, W. Majewski et al. Multiparametric scaling of diffraction intensities. Acta Crystallogr A 59 (Pt 3), 228 (2003).
³¹ G. J. Kleywegt and T. A. Jones. Detection, delineation, measurement and display of cavities in macromolecular structures. Acta Crystallogr D Biol Crystallogr 50 (Pt 2), 178 (1994).
³² A. C. Mustilli, S. Merlot, A. Vavasseur et al. Arabidopsis OST1 protein kinase mediates the regulation of stomatal aperture by abscisic acid and acts upstream of reactive oxygen species production. Plant Cell 14 (12), 3089 (2002).
³³ S. D. Yoo, Y. H. Cho, and J. Sheen. Arabidopsis mesophyllprotoplasts: a versatile cell system for transient gene expression analysis. Nature protocols 2 (7), 1565 (2007).

What is claimed is:

1. A method of improving stress resistance in a plant, the method comprising contacting the plant with a compound of formula I, formula II or formula III 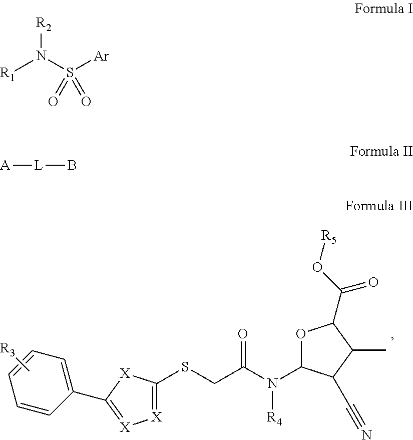
wherein R₁ is a C₁₋₆ branched or straight alkyl group which is optionally and independently substituted with up to 3 of phenoxy, benzyloxy, R′ or R″, each of which is optionally and independently substituted with up to 3 of R′″;
Ar is a phenyl or naphthyl group, which is optionally and independently substituted with up to 3 of R₆;
Each R₆ is independently R′ or R″; or two adjacent R₆ substituents, together with the atoms to which they are attached, form a C₃₋₈ cycloalkyl or heterocycloalkyl ring, which is optionally and independently substituted with up to 3 of R′ or R″;
A and B are each independently phenyl, cyclohexyl or cyclohexenyl, each of which is optionally and independently substituted with up to 4 of R′ or R″;
L is a divalent linear saturated or unsaturated C₄ aliphatic chain which is optionally and independently substituted with R′ or R″, and wherein up to 4 carbon units of the C₄ aliphatic chain are optionally and independently replaced with —C(O)—, —C(S)—, —NR″— or —O—;
R₃ is R′ or R″;
R₂, R₄, and R₅ are each independently R″;
Each X is independently N, NH, N—C₁₋₄ alkyl, O, S, or CH;
Each R′ is independently hydrogen, halo, oxo, —OR″, —C(O)R″, —C(O)OR″, —C(O)NR″₂, —SR″, —S(O)₂R″, —S(O)₂OR″, or —S(O)₂NR″₂;
Each R″ is independently absent, hydrogen, C₁₋₆ alkyl, C₆ heteroaryl or heterocycloalkyl, each of which is optionally and independently substituted with up to 3 of R′″;
Each R′″ is independently hydrogen, halo, oxo, OH, NH₂, NO₂, COOH, C₁₋₄ alkyl, or C₁₋₄ haloalkyl;
with the proviso that the compound is not pyrabactin:

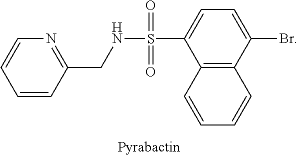 [SMILES: O=S(=O)(NCc1ccccn1)c1ccc(Br)c2ccccc12]

2. The method of claim 1, wherein R₁ is heteroaryl alkyl, alkoxy carbonyl alkyl, heterocycloalkyl alkyl, alkylthio (carboxy)alkyl, aryl alkoxycarbonyl alkyl, alkylamino carbonyl (carboxy)alkyl, dialkylamino carbonyl (carboxy)alkyl or aryloxy alkyl.

3. The method of claim 2, wherein R₁ is

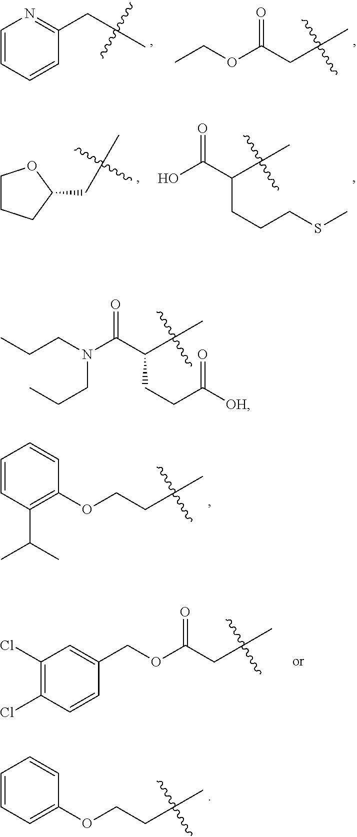

4. The method of claim 1, wherein Ar is phenyl, alkylphenyl, naphthyl, halonaphthyl, alkylnaphthyl, or a substituent having the formula Ar′

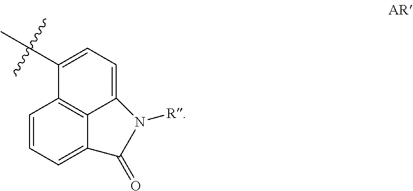

5. The method of claim 4, wherein Ar is

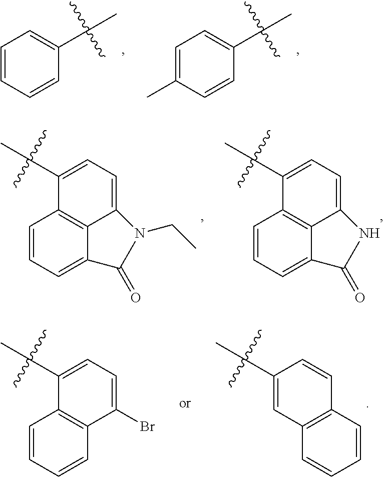

6. The method of claim 1, wherein A and B are each independently alkyl (carboxy)phenyl, halophenyl, aminosulfonyl phenyl, alkoxy phenyl, alkylphenyl, dihalophenyl, acetylphenyl, dioxo dialkyl cyclohexane or hydroxyl oxo dialkyl cyclohexene.

7. The method of claim 6, wherein A and B are each independently selected from

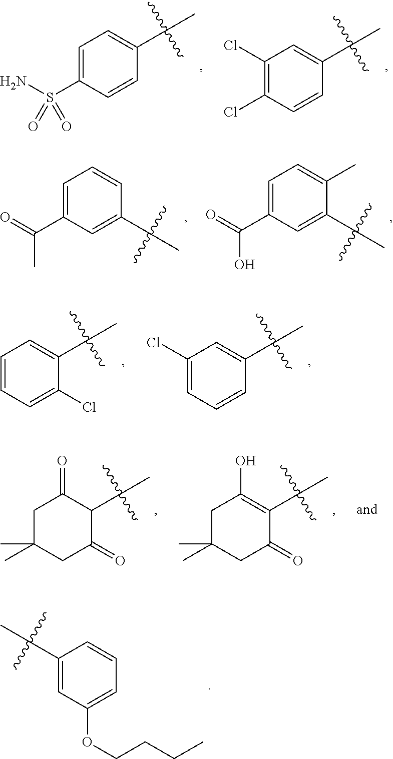

8. The method of claim 1, wherein L is a divalent linear saturated or unsaturated C₄ aliphatic chain which is optionally substituted with R′ or R″, and wherein up to 4 carbon units of the C₄ aliphatic chain are optionally and independently replaced with —C(O)—, —C(S)—, or —NH—.

9. The method of claim 8, wherein two carbon units are replaced with —NH—, one carbon unit is replaced with —C(O)—, and one carbon unit is replaced with —C(S)—.

10. The method of claim 9, wherein L is

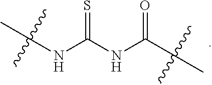 [SMILES: *NC(=S)NC(*)=O]

11. The method of claim 1, wherein L is a divalent linear unsaturated C₄ aliphatic chain which is optionally substituted with R′ or R″, and wherein one carbon unit of the C₄ aliphatic chain is optionally and independently replaced with —NH— or —N═ and one carbon is optionally replaced with C(O).

12. The method of claim 11, wherein L is

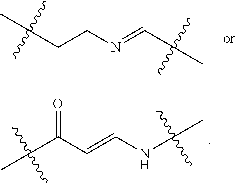 [SMILES: */C=N/CC* ; *N/C=C/C(*)=O]

13. The method of claim 1, wherein
R₃ is C₁₋₆ alkoxy;
each X is N or O;
R₄ is hydrogen; and
R₅ is C₁₋₆ alkyl.

14. The method of claim 13, wherein
R₃ is ethoxy; and
R₅ is ethyl.

15. The method of claim 1, wherein the compound is selected from

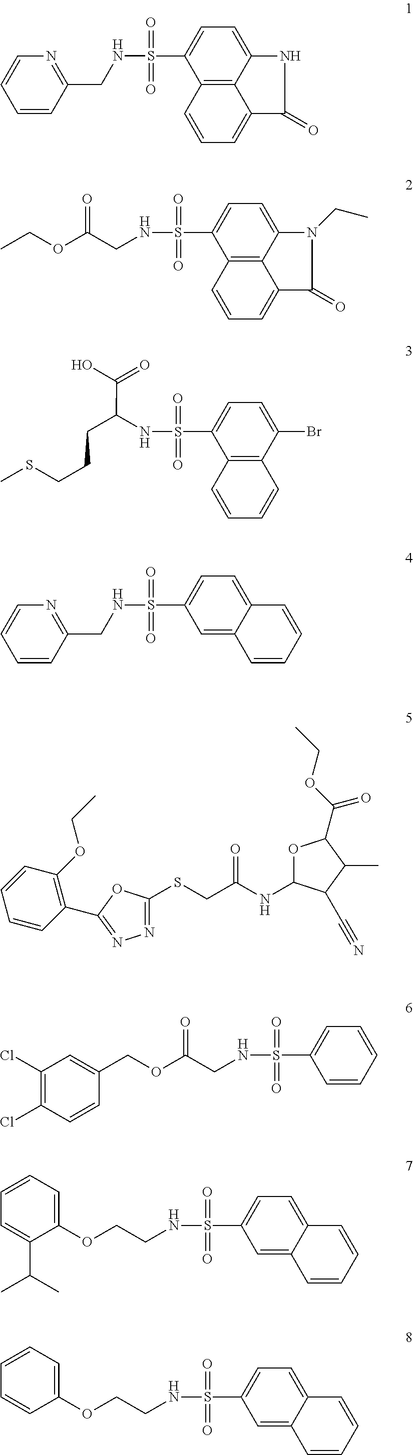

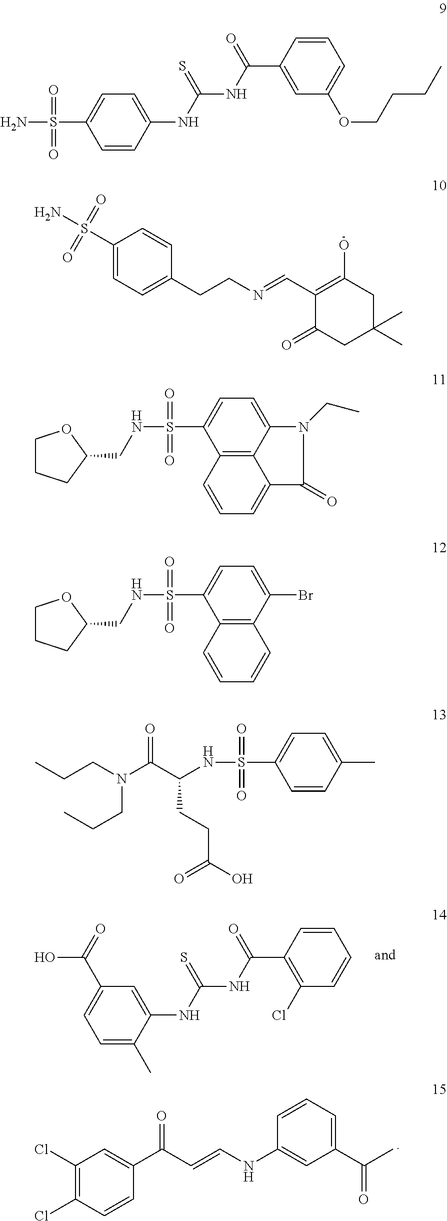

16. The method of claim 1, wherein the plant is contacted directly with the compound or a composition or formulation comprising the compound.

17. The method of claim 1, wherein the plant comprises a seed, and wherein the seed is contacted directly with the compound or a composition or formulation comprising the compound.

18. The method of claim 1, wherein the plant comprises a locus, in which the plant grows or will grow, and wherein the locus is contacted directly with the compound or a composition or formulation comprising the compound.

19. The method of claim 1, wherein the stress is an abiotic stress.

20. The method of claim 19, wherein the abiotic stress is selected from the group consisting of drought, high salinity, osmotic stress, heat or cold.

21. The method of claim 20, wherein the osmotic stress is high sugar concentration.

22. The method of claim 20, wherein the stress is drought.

23. The method of claim 20, wherein the stress is high salinity.

24. The method of claim 1, wherein the plant is a land plant.

25. The method of claim 24, wherein the land plant is a moss or a fern.

26. The method of claim 1, wherein the plant is an underwater plant.

27. The method of claim 26, wherein the underwater plant is green algae.
